(19) United States
(12) Patent Application Publication
(10) Pub. No.: US 2006/0064089 A1
(43) Pub. Date: Mar. 23, 2006

(54) Bone screw closure having domed rod engaging surface

(76) Inventor: Roger P. Jackson, Prairie Village, KS (US)

Correspondence Address: John C. McMahon, PO Box 30069, Kansas City, MO, 64112, US

(21) Appl. No.: 10/935,411

(22) Filed: Sep. 7, 2004

(51) Int. Cl. A61F 2/30

(52) U.S. Cl. 606/61

(57) ABSTRACT

An implant for insertion on or into a bone of a patient and for receiving a rod member in an open channel formed between two arms of a head thereof. A closure for closing the channel and capturing the rod in the open channel. The closure including an axially projecting dome that is positioned to engage and abut against the rod in use. The closure also including guide and advancement structure that joins with mating guide and advancement structure in the bone screw head. The guide and advancement structure preferably resists splaying of the arms during installing of the closure. Most preferably, the guide and advancement structure is helically wound mating and interlocking flange form structure or reverse angle thread form structure.

### BACKGROUND OF THE INVENTION

The present application is directed to a closure for closing a channel in a rod receiving open spinal implant, especially a bone screw, so as to capture and fixedly position the rod relative to the spinal implant, that is used to anchor the rod to the spine.

Bone screws are utilized in many different medical procedures to secure implants to patient's bones. Typically, the bone screw is fixedly positioned within a bone and a second implant such as a rod or a rod-like structure is secured to the bone screw. A common use of such implants is to strengthen or replace one or more vertebral bones or to correct curvature of vertebrae within the spine. In such processes, the bone screws are conventionally implanted in various selected vertebrae along the spine and connected by rods or other implants which provide support to the spine where the spine has been damaged or weakened due to accident, disease or genetic predisposition.

Bone screws utilized for this purpose may be of two types. In a fixed or monoaxial type of bone screw, the head of the bone screw that receives the second implant is permanently fixed relative to a threaded shank that screws into the bone. A second type of bone screw is a polyaxial bone screw where the shank rotates relative to the head during initial installation stages to allow the head to be positioned and thereafter locked in position. Both types of bone screws are utilized in spinal surgery and may be used in combination or separately. However, the occurrence of use of the polyaxial bone screw is much greater than the monoaxial bone screw. Nevertheless, an overall system of the implants should be able to take advantage of either type of bone screw.

Each of the types of bone screws typically has a head which receives a rod-like implant in an open channel. It is possible to have bone screws where the channel is closed, but those type of bone screws are not the subject of the present application and are difficult to use since a rod must be threaded through some kind of opening for the rod, which makes them relatively difficult to use and, consequently, the number of such bone screws is significantly lower than the type having an open channel. The open channel allows the rod-like implants to be placed within the channel from above by hand or be urged there by some type of tooling. In such bone screws having an open head, there must be some type of closure for closing the head so as to capture the rod within the head and, also preferably to urge the rod into a seated and locked position relative to the head.

Certain types of prior art closures for this purpose have included nuts which essentially go about the outside of the bone screw head and abut against the rod as the nuts are tightened down on the head. Furthermore, closures have been utilized that slide in from the side and are thereafter locked in place by a set screw or some similar structure. A third type of closure is a plug-like structure having a closure body that lies between opposite or opposed arms of the head and abuts against the rod. The closures of the present application are generally of the later type of closures.

Certain prior art plug type closures have been threadably received between the opposed arms of the bone screw head using conventional V-shaped thread forms which has resulted in a significant amount of radially outward pressure or force being applied to the arms of the bone screw head. Such outward force may result in splaying of the arms after which the closure becomes loose which may either result in a failure of the implant by allowing the rod to slip relative to the bone screw or the closure may even come completely out of the head of the bone screw for total failure of the implant. In either case, the results are entirely unsatisfactory. Nevertheless, a substantial amount of torque is required to seat the plug against the rod which in turn seats the rod in the head channel so as to prevent relative motion between the rod and the bone screw. Consequently, the need to highly torque the plug functions counter to the need to prevent the arms from splaying. In order to help relieve this problem, certain of the prior art has added structure to the rod engaging lower surface of the closure. In particular, the most common structure has been a central or axial point which has been designed to penetrate into the rod and help lock the rod into place. The second type of structure for this purpose has been a ring-like structure having a sharpened lower edge that is also centered axially with respect to the closure and which cuts into the rod at a radius spaced from the point to give additional penetration into the rod. A third attempt at holding the rod in place at lower torquing pressure has been the use of knurling on the bottom surface of the closure which is designed to cut into and help secure the rod in place.

Absent the use of an external nut (which undesirably increases the profile of the implant) to prevent splaying in combination with an internal closure, it has been difficult in the prior art to assure that sufficient torque can be applied to the closure to lock the rod in place relative to the head and yet also be sure that no splaying of the arms occurs. Consequently, a need exists for a closure having a rod engaging surface that is suitable for engaging the rod and that can be torqued to a sufficient torque to ensure that no slippage occurs between the rod and the head of the bone screw or other spinal implant while simultaneously ensuring that the opposed arms of the head do not splay.

### SUMMARY OF THE INVENTION

The present invention is directed to a spinal implant, especially a bone screw implant for use in humans and especially for use in surgical procedures wherein it is required to secure rods or rod-like implants in vertebrae or other boney structure of a human.

The bone screws of the present invention include a threaded shank which is adapted to be fixedly secured in a bone and an upstanding head which has an open channel that is adapted to receive a rod or rod-like member. The bone screw may be of either a fixed, monoaxially type or an initially rotatable polyaxially type wherein the shank is rotatable relative to the head until fixed at some point during surgery. The head includes two spaced apart arms which form the open channel therebetween and receive a closure between the arms to close the upper end of the channel. The closure captures the rod or rod-like member and preferably also locks the rod in a fixed relationship to the bone screw.

The closure comprises a cylindrical body having a helical wound guide and advancement structure on the radially external surface thereof. The guide and advancement structure may be any helical type structure that allows the closure to be rotated and advanced toward the rod and urged against the rod under torque. The guide and advancement structure has a mating guide and advancement structure that comprises two sets of receivers that are located in opposed or facing relationship in the arms. Preferably, the guide and advancement structure either urges the arms inwardly, as in the case of a reverse angle thread form, or alternatively, radially locks the arms in place relative to the closure during positioning of the closure within the channel, as in the case of a flange form, so as to lock the arms in place and prevent splaying thereof. Preferred guide and advancement structure of this type is a helical wound reverse angle thread form or alternatively, a helically wound flange form. The closure also includes a domed lower rod engaging surface. The dome may be radiused with a radius of generation that is larger or smaller than the radius of the cylinder forming the body of the closure, but preferably larger. The dome may also have a curved surface other than a spherical surface forming a complex curve with a central apex or may have a spherical surface along the axis and feather out toward the edges or the like. The dome on the bottom of the closure is in any event centered so that the furthest extension of the dome or apex is along the central axis of the body of the closure and also at the front or leading end of advancement as the closure is inserted into the head of the bone screw.

The closure may include various types of structure for driving the closure initially and/or removing the closure should removal be necessary. For example, the closure may include a break off head that has a socket type exterior for use in driving and torquing the closure to a preselected torque at which point the break off head breaks away from the body leaving the body of the closure between the arms of the head. The body may also be driven by other types of structures such as a centrally located aperture having, for example, a hexagonal pattern commonly referred to as an Allen type drive or one commonly referred to as a Torx type drive. The closure body may also have alternatively, spaced apertures designed for receiving a tool to be utilized to drive the device or a slot for receiving a screw driver type tool. Certain of the previously described structure may also be utilized to remove the closure, such as the Allen, Torx and spaced apart apertures or additional structure such as a step down bore may be utilized to be engaged with an easy out or the like to remove the closure should removal be necessary to reposition the implant in some manner.

### OBJECTS OF THE INVENTION

Therefore, the objects of the present invention are: to provide a closure for use in conjunction with an open channel spinal implant, particularly a bone screw, and especially a vertebral bone screw, for closing a channel in the head of the bone screw and for securing a rod or rod-like member in the head and further lockably positioning the rod relative to the head; to provide such a closure having a domed rod engaging surface; to provide such a closure having guide and advancement structure that resists splaying of upstanding and opposed arms of the head of the bone screw; to provide such a closure having a reverse angle thread as guide and advancement structure; to provide such a closure having an interlocking helical flange as a guide and advancing structure; to provide such a closure adaptable for use with a variety of driving and removal structures; to provide such a closure that can be used in a comparatively reduced volume or low profile implant and torqued to a comparatively high torque relative to the prior art; and to provide such a closure which is easy to use, comparatively inexpensive to produce and especially well adapted for the intended usage thereof.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is a perspective view of a first bone screw closure in accordance with the present invention.

FIG. 2 is a side elevational view of the first closure.

FIG. 3 is a top plan view of the first closure.

FIG. 4 is a bottom plan view of the first closure.

FIG. 5 is a fragmentary, exploded and perspective view of the first closure in conjunction with a bone screw and rod on a reduced scale and prior to capture of the rod with the closure.

FIG. 6 is a fragmentary side elevational view of the bone screw and first closure with the closure capturing the rod.

FIG. 7 is a fragmentary side elevational view of the bone screw and first closure subsequent to break away of a driving head of the closure.

FIG. 8 is an enlarged cross sectional view of the first closure lockably securing a rod within the bone screw, taken along like 8-8 of FIG. 5.

FIG. 9 is a perspective view of a second closure for a bone screw in accordance with the present invention.

FIG. 10 is a side elevational view of the second closure.

FIG. 11 is a top plan view of the second closure.

FIG. 12 is a bottom plan view of the second closure.

FIG. 13 is a fragmentary, exploded and perspective view on a decreased scale of the second closure utilized in conjunction with the second bone screw prior to capture of a rod in the second bone screw.

FIG. 14 is a side elevational view of the second bone screw and second closure showing the second closure just prior to placement in the second bone screw and a tool for driving and rotating the second closure.

FIG. 15 is a fragmentary side elevational view of the second bone screw showing the second closure positioned within the second bone screw and with a driving tool removed therefrom.

FIG. 16 is a fragmentary cross sectional view on an enlarged scale of the second bone screw and second closure, taken along line 16-16 of FIG. 13.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

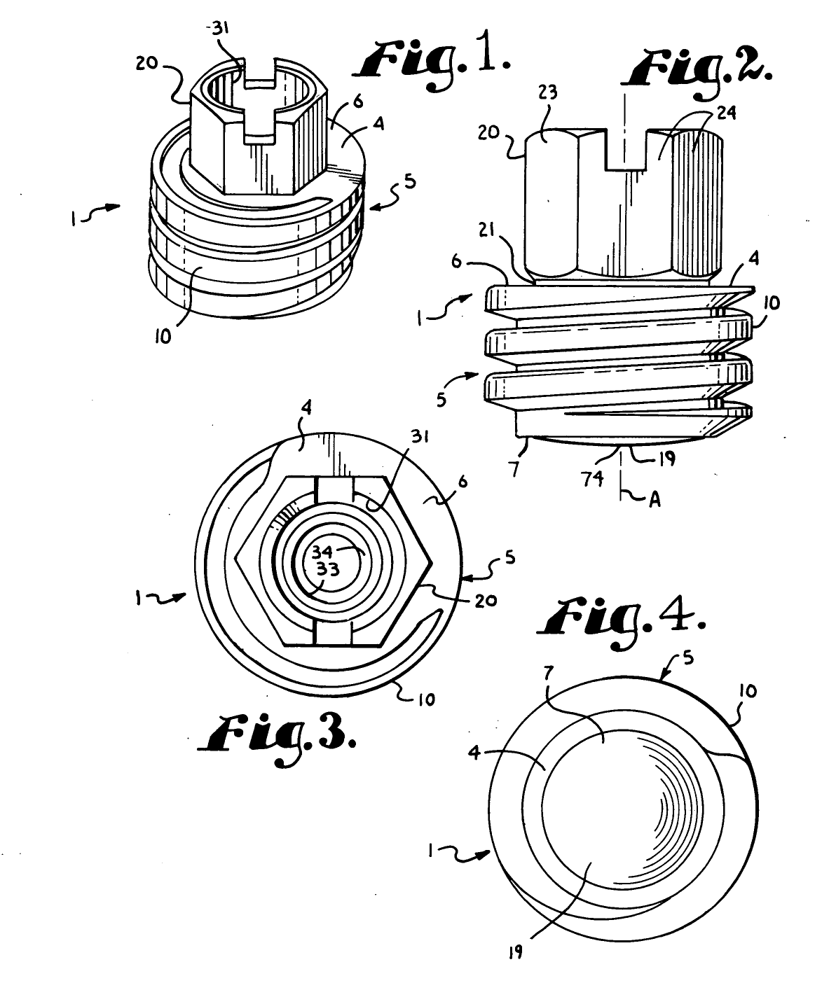

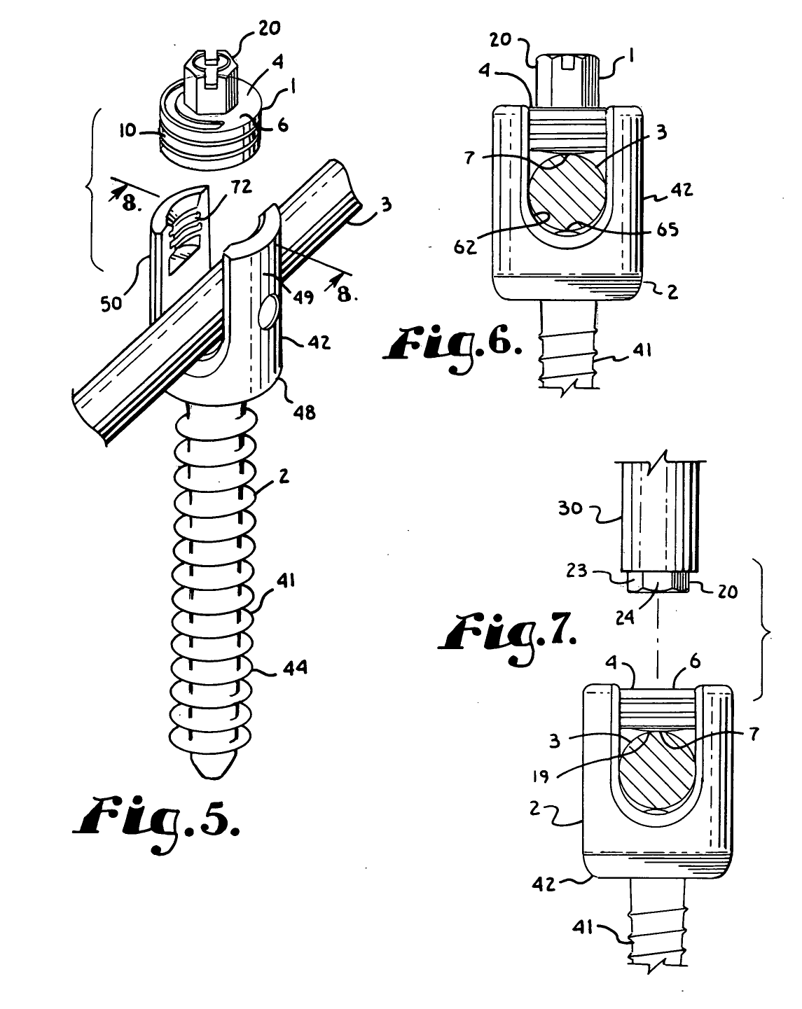

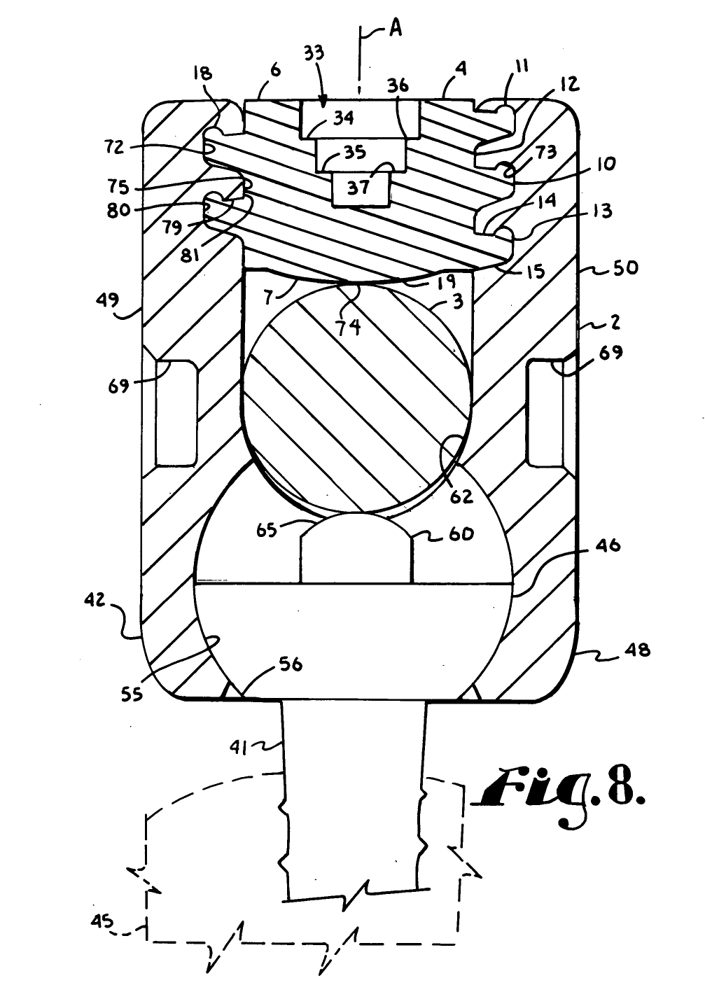

The reference number 1 generally designates a bone screw closure that is illustrated in FIGS. 1 through 8 that is used within a bone screw 2 to capture and secure a rod or rodlike member 3. The closure 1 includes a cylindrical body 4 having a radially outer surface 5, a top or trailing surface 6 and a lower or lead surface 7.

Helically wound about the body outer surface 5 is a first guide and advancement structure 10. The guide and advancement structure 10 includes a flange form 11 which operably joins with a mating structure, discussed below. The flange form 11 includes a root 12 and a crest 13. Furthermore, the flange form 11 also has a trailing surface 14 and a leading surface 15 which are relative to the forward movement of the closure 1 as it is rotated clockwise about a central axis A and joined with the bone screw 2. Located on the trailing surface 14 or the leading surface 15 or both is a projection which protrudes rearwardly or frontwardly with respect to the width of the flange form 11 at the root 12 and which interlocks with a guide and advancement mating structure, described below.

In the illustrated embodiment, the flange form 11 has a protrusion 18 that projects rearwardly from the trailing surface 14. The flange form 11 utilized in accordance with the present invention may be any structure which effectively locks the closure 1 to the structure within which it is set so as to prevent splaying of the structure upon which mating guide and advancement structure is mounted. Various flange form structures which can be used alternatively are illustrated in applicant's U.S. Pat. No. 6,726,689, which is incorporated herein by reference.

The closure lower surface 7 has a dome 19 located thereon. The dome 19 extends greatest from the body 4 along the axis A. The dome 19 in the present embodiment is spherical in shape and, in particular, is a partial sphere that has a uniform or constant radius of generation.

However, it is foreseen that in certain embodiments the radius may vary depending upon the needs and desires of the particular structure and the dome 19 may have shape that is only partly a spherical curved surface or some other shape. The dome 19 may be simple a curved surface that allows greatest projection along the axis. That is, the dome surface 7 could be radiused at the location of greatest projection and feathered along the periphery so as to not have a continuous uniform radius of generation throughout, but rather a continually changing radius of generation along at least the length thereof. Preferably, the dome 19 is smoothly curved where the dome 19 intersects with axis A.

The closure 1 also includes a break off head 20 that is secured to the body 4 by a break off region 21 that is designed to allow the head 20 to break from the body 4 at a preselected torque, for example, 150 inch pounds. The break off head 20 has an external radial outward surface 23 with six planar facets 24 so as to form a structure designed to be received within a socket of a driving type tool 30 with a similar receiving shape. The break off head 20 has a central tool receiving bore 31. At the bottom of the bore 31 is a step down region 33 having two separate steps 34 and 35 that are sized and shaped to provide evenly spaced shoulders 36 and 37 that provide edges are suitable for engagement by an easy out tool (not shown) for purposes of removal.

The closure 1 is received in the bone screw 2. The illustrated bone screw 2 has a shank 41 and a head 42. The bone screw shank 41 has a helical wound thread 44 thereon and is designed to be threadedly received within a bone 45 of a patient. The shank 41 is received and captured in a capture ring 46 such that once joined, the shank 41 and ring 46 become preferably permanently secured to one another. The head 42 includes a body 48 and a pair of upstanding arms 49 and 50. The head 42 also has an internal generally hemispherically shaped chamber 55 and a lower aperture 56. An upper end of the shank 41 is received through the aperture 56 while the capture ring 46 is received within the chamber 55 and initially is slidably and rotatably received in the chamber 55, so that the shank 41 is initially rotatably relative to the head 42. The shank 41 has an upstanding projection 60 that protrudes axially upward therefrom. When received in the chamber 55, the projection extends upwardly through the chamber 55. The head 42 has a channel 62 that is located between the arms 49 and 50. The projection 60 extends into the channel 62. During use, the rod 3 is located or positioned within the channel 62 and secured in place therein by the closure 1. In particular, the projection 60 engages the rod 3, as seen in FIG. 8 when the rod 3 is in the channel 62. The closure 1 is rotatably advanced into a pair of mating guide and advancement structures 72 and 73 on the arms 49 and 50 until the dome 19 engages the rod 3, especially in line with the axis A. The mating guide and advancement structures 72 and 73 include a mating flange form 75 having a counter extending and locking projection 79 and also having a foot 80 and a crest 81. The dome 19 especially engages the rod 3 at an apex 74 as seen in FIG. 8. The closure 1 is torqued until a preselected pressure is reached at which point the closure 1 at the apex 74 abuts the rod 3 which in turn is urged toward but not completely to the bottom of the channel 62. In turn, the rod 3 braces against the shank projection 60 which urges the capture ring 46 to fixedly seat in the chamber 55. Thereafter, the head 42 is no longer rotatable relative to the shank 41, but rather is locked in position.

Tool gripping apertures 69 are located on opposite sides of the head 48 for gripping by tools (not shown) for various purposes.

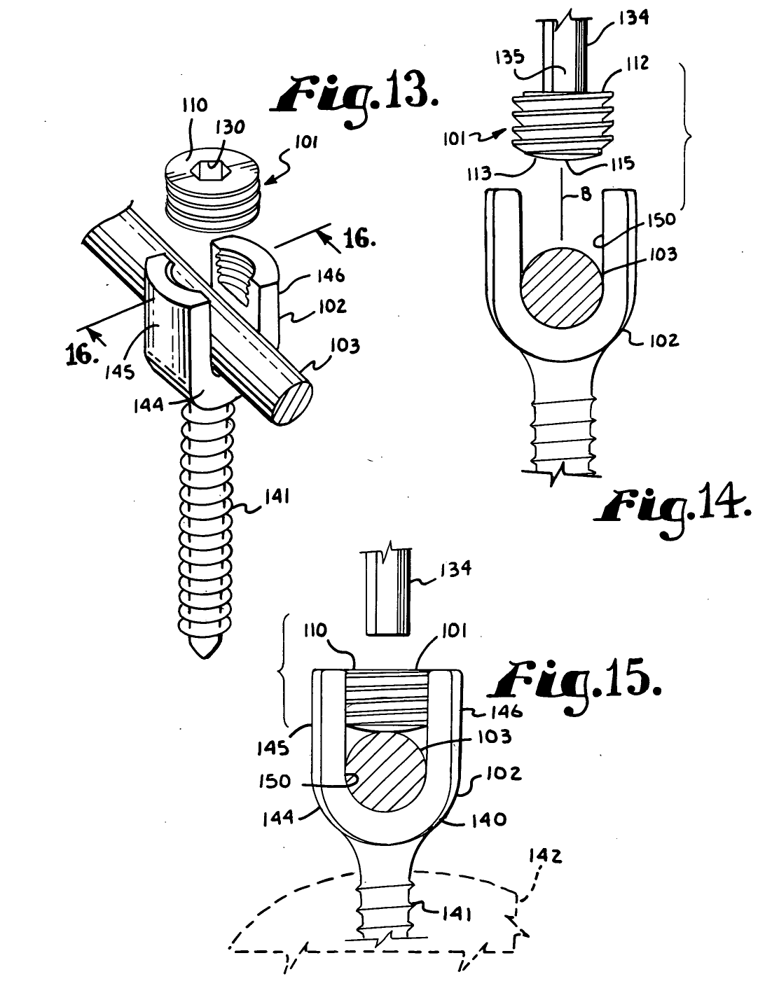

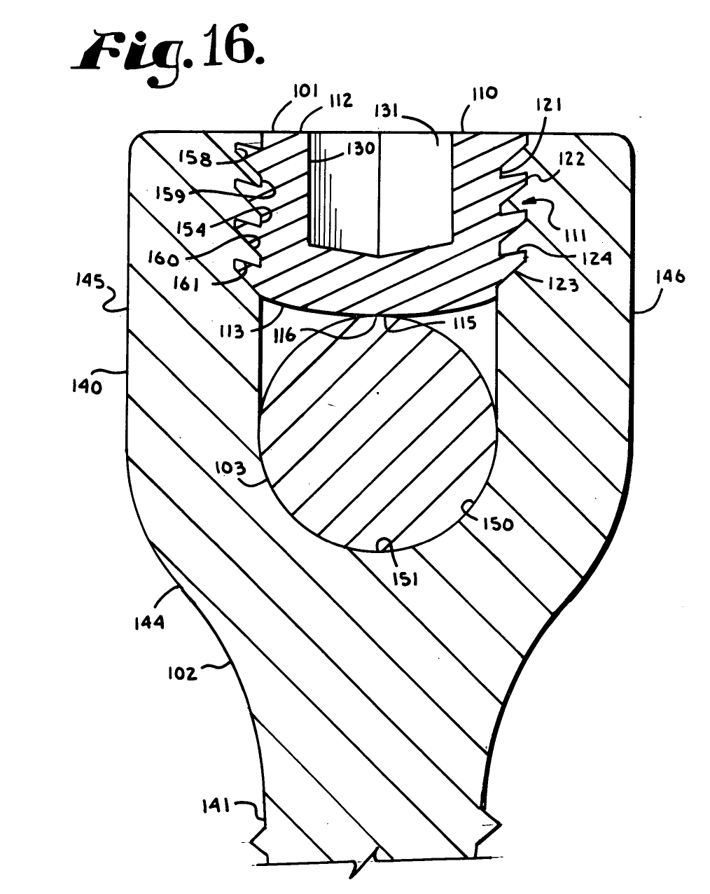

The reference numeral 100 generally designates a second embodiment of a closure in accordance with the present invention which is illustrated in FIGS. 9 to 16. The closure 100 is utilized with a bone screw 102 and a rod 103.

The closure 100 has a generally cylindrical or plug shaped body 110 with a cylindrically shaped radial outer surface 111 and a central axis of rotation B. The closure 100 has an upper, top or trailing surface 112 and a lower, bottom or lead end 113.

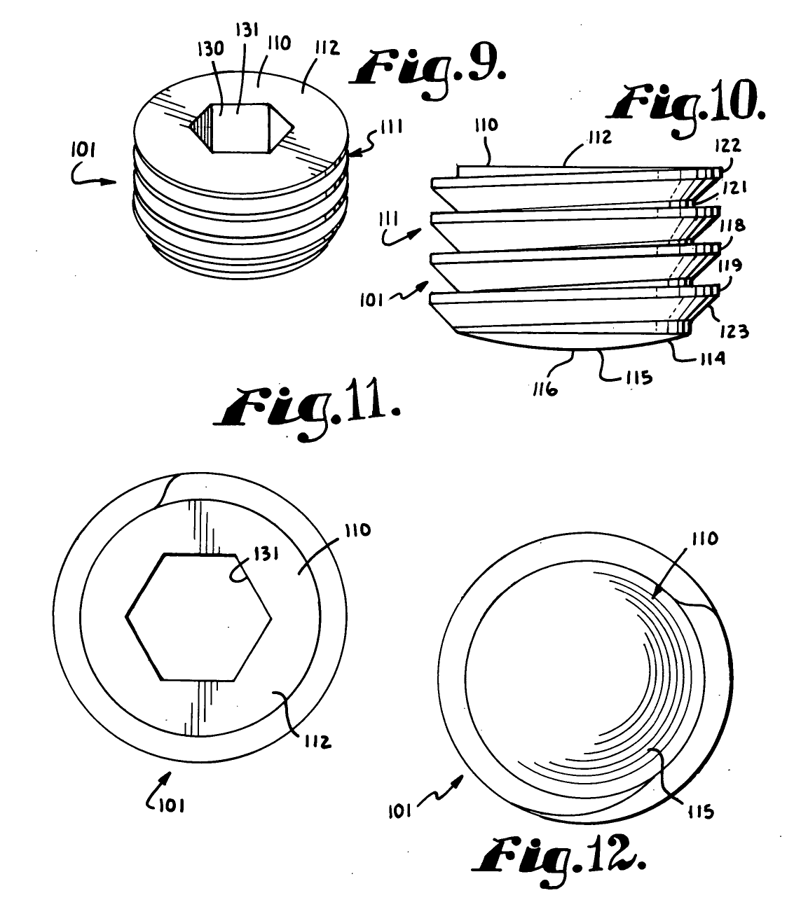

Located on the lead end 113 is a convex shaped region or dome 115 that projects outwardly from the body 110 along the axis B (downwardly in FIG. 10) and has an apex 116 whereat the dome 115 intersects the axis B.

In this embodiment, dome 115 covers the entire bottom end 113 of the closure 100. In contrast, the dome of the prior embodiment covered only a portion of the lower surface of the closure 1. It is foreseen that domes in accordance with the invention may cover more or less of the bottom surface and may vary in radius of generation or curvature. However, in a spherical dome having a radius of generation that is substantially greater than the radius of the closure surface 111 and which is located to project in the range of 5% to 15% beyond the length of the cylindrical body 110. It is preferred that the dome 115 be smooth and convex whereat the axis B intersects with the dome 115 and not pointed. However, in certain embodiments, it is foreseen that the dome could be at least partially covered with knurling or the like to provide additional gripping during usage.

Located on the closure cylindrical surface 111 is a guide and advancement structure 118 which in the present embodiment is a helical wound reverse angle thread form 119. The guide and advancement structure 118 acts cooperatively, as described below with the bone screw 102 to allow the closure 100 to be inserted into and rotated relative to the bone screw 102 and to guide and advance the closure 100 along the axis B as the closure 100 is rotated clockwise or to reverse direction when rotated counterclockwise. The guide and advancement structure 118 resists splaying in the bone screw 102 as forces applied to the closure 100 are conveyed by the reverse angle thread form 119 during application of clockwise rotational torque into a downward axial force and inwardly directed radial force. It is foreseen that other types of guide and advancement structure could be utilized. For example, a buttress thread form or a square thread form may be effectively used which theoretically has little or no radially outward directed forces, especially if the arms are thickened to resist splaying. Other thread forms may also be used with the dome 115.

The thread form 119 has a root 121 and a crest 122. Further, the thread form 119 has a lead surface 123 and a trailing surface 124 (described relative to the position thereof during insertion of the closure 100 into the bone screw 102). In a reverse angle thread, the trailing surface 124 from the root 121 to the crest 122 extends at an angle rearwardly from a perpendicular line relative to the axis B. Normally, the trailing surface 124 is at an angle between 1 and 20 degrees relative to such a perpendicular.

Located in the closure top surface 112 is an aperture 130 that is centrally located and axially extending. The aperture 120 is faceted with six equal walls to form an inner faceted wall 131 sized and shaped to receive an Allen style driving tool 134 with a mating surface 135 thereon.

The bone screw 102 includes a head 140 and a threaded shank 141. The bone screw 102 is of a fixed headed bone screw type as opposed to the polyaxial type of the first embodiment wherein the head can rotate relative to the shank prior to being locked in place by torquing of the closure. The shank 141 of the bone screw 102 is operably screwed into a bone 142, such as a vertebral body in the spine of a patient.

The head 140 has a body 144 with a pair of upstanding spaced arms 145 and 146. The body 144 in conjunction with the arms 145 and 146 form between and define a rod receiving channel 150 having a width approximately equal to the diameter of the rod 103 and a seat 151 which snugly receives the rod 103 during installation, as seen in FIG. 16.

Located on inwardly facing surfaces of each of the arms 145 and 146 are mating guide and advancement thread forms 154 and 155 respectively that are sized and shaped to receive the closure thread form 119 during installation and rotation of the closure 100 between the arms 145 and 146. Each of the thread forms 154 and 155 have a root 158 and a crest 159, as well as a first mating surface 160 that mates with the closure lead surface 123 and a second mating surface 161 which mates with the closure trailing surface 124.

It is foreseen that a closure of the present invention may have a wide range of structures for installing, driving and removing the closure. In the first embodiment, the break off head 20 is utilized for driving and torquing the closure 1 in the bone screw 2, while the step down bore region 33 may be effectively used with an easy out (not shown) for removal. In the second embodiment of the closure 100, the Allen tool receiving aperture 130 receives the tool 134 for both installation and removal. It is foreseen that structures including spaced apertures or other structure may effectively be used to aid in installing or removing the closure from the head of the bone screw.

In use, the bone screw 102 is screwed into and secured to a bone 142, such as is shown in FIG. 15. The rod 103 is placed in the channel 150 and the closure 100 is inserted into the channel 150 by aligning the thread form 119 with the mating thread forms 154 and 155 on the head 140 and rotating clockwise using the tool 134 to rotate the closure 100 and drive the apex 116 against the rod 103, as shown in FIG. 15, until a desired torque is achieved. The tool 134 is then removed. If it is later desired to remove the closure 100, the tool 134 is reinserted into the aperture 130 and the process is reversed.

The apex 116 of the dome 115 abuts against and secures the rod 3 in a fixed position both relative to the bone screw 2 and the closure 1. Because the guide and advancement structure of both this embodiment and the first illustrated embodiment resist splaying of the arms 145 and 146 by directing forces inward in the case of the present embodiment due to the reverse angle thread and by radially locking together the closure and arms in the first embodiment using the flange form, greater torque can be applied to the closure 1 and 100 in comparison to the prior art which allows the smooth surfaced domes 19 and 115 to be effective in fixing the rods relative to the respective heads and closures.

It is to be understood that while certain forms of the present invention have been illustrated and described herein, it is not to be limited to the specific forms or arrangement of parts described and shown.

What is claimed and desired to be secured by Letters Patent is as follows:

1. A closure adapted for use in closing a rod receiving channel of a bone screw; said closure comprising:
a) a body having a central axis of rotation and a radial outer surface;
b) a dome centered on said axis of rotation and extending outwardly from a leading end of said body; and
c) a splay resistant guide and advancement structure on said body outer surface.

2. The closure according to claim 1 wherein:
a) said dome has a partial spherical shape having a radius of generation.

3. The closure according to claim 2 wherein:
a) said dome radius of generation is greater than a radius of said closure body outer surface.

4. The closure according to claim 1 wherein:
a) said dome has an apex that is located along said axis and said dome is smoothly curved whereat said dome intersects said axis.

5. The closure according to claim 1 wherein:
a) said dome extends axially outward from said body in the range of from 5 to 15 percent of an axial length of said body.

6. The closure according to claim 1 wherein:
a) said guide and advancement structure is a helical wound flange form.

7. The closure according to claim 1 wherein:
a) said guide and advancement structure is a helical wound reverse angle thread form.

8. The closure according to claim 1 including:
a) a break off installation head.

9. The closure according to claim 1 including:
a) removal structure adapted to mate with a removal tool.

10. A bone screw and closure assembly adapted to be used with a rod member comprising a bone screw and closure wherein:
a) said bone screw includes a head and a shank;
b) said head having a body with a pair of upstanding and spaced arms forming a channel sized and shaped to receive the rod member;
c) each of said arms including guide and advancement structure on facing surfaces thereof;
d) said shank being adapted to be implanted in a bone of a patient;
e) said closure having a body with a cylindrical shaped radially outer surface and an axis of rotation;
f) a mating guide and advancement structure helically wound on said body outer surface; said mating guide and advancement structure operably joining with said arm guide and advancement structures to guide said closure under rotation to close said channel while capturing the rod under rotation; and
g) a dome extending from said body along said axis from a leading end of the body.

11. The assembly according to claim 10 wherein:
a) said dome has a partial spherical shape having a radius of generation.

12. The assembly according to claim 11 wherein:
a) said dome radius of generation is greater than a radius of said closure body outer surface.

13. The assembly according to claim 10 wherein:
a) said dome has an apex that is located along said axis and said dome is smoothly curved whereat said dome intersects said axis.

14. The assembly according to claim 10 wherein:
a) said dome extends axially outward from said body in the range of from 5 to 15 percent of an axial length of said body.

15. The assembly according to claim 10 wherein:
a) said guide and advancement structure is a helical wound flange form.

16. The assembly according to claim 10 wherein:
a) said guide and advancement structure is a helical wound reverse angle thread form.

17. The assembly according to claim 10 including:
a) a break off installation head.

18. The assembly according to claim 10 including:
a) removal structure adapted to mate with a removal tool.

19. The assembly according to claim 10 wherein:
a) said bone screw is a polyaxial bone screw.

20. The assembly according to claim 10 wherein:
a) said bone screw head is fixedly and rigidly attached to said bone screw shank.

21. In a closure for an open spinal implant having a cylindrical body with an axis of rotation and adapted to close a channel between spaced arms of the implant, the improvement comprising:
a) said closure having a partial spherical dome extending from a leading end of the body and having an apex positioned so as to engage a rod in the channel and centered on said axis.

22. The closure according to claim 21 wherein:
a) said dome is smoothly convex at an intersection of the dome with the axis and a radius of generation the dome is greater than the radius of a radial outer surface of said closure body.

23. In a closure having a cylindrical body with an axis of rotation for closing a channel in a head of a bone screw between a pair of arms and capturing a rod in the channel formed between arms of the bone screw; the improvement comprising:
a) a dome centered on the axis of rotation and having an apex located on a leading end of said body so as to be adapted to engage the rod during use; and
b) a splay resistant guide and advancement structure on said body adapted to join with a mating guide and advancement structure on said bone screw during installation and adapted to resist splaying of the arms.

24. A closure adapted for use in closing a rod receiving channel of an open spinal implant; said closure comprising:
a) a body having a central axis of rotation and a radial outer surface;
b) a dome centered on said axis of rotation and extending outwardly from a leading end of said body; and
c) a guide and advancement structure on said body outer surface.

25. The closure according to claim 24 wherein:
a) said guide and advancement structure is a helical wound buttress thread form.

26. The closure according to claim 24 wherein:
a) said guide and advancement structure is a helical wound square thread form.
